(19) United States
(12) Patent Application Publication
(10) Pub. No.: US 2022/0292298 A1
(43) Pub. Date: Sep. 15, 2022

(54) METHOD OF BUILDING MODEL FOR ESTIMATING LEVEL OF PSYCHOLOGICAL SAFETY AND INFORMATION PROCESSING DEVICE

(71) Applicant: FUJITSU LIMITED, Kawasaki-shi (JP)

(72) Inventor: Atsuya Suzuki, Kawasaki (JP)

(73) Assignee: FUJITSU LIMITED, Kawasaki-shi (JP)

(21) Appl. No.: 17/563,358

(22) Filed: Dec. 28, 2021

(30) Foreign Application Priority Data
Mar. 15, 2021 (JP) 2021-041880

(51) Int. Cl. G06K 9/62; G06V 30/19; A61B 5/16

(52) U.S. Cl. CPC G06K 9/6235; G06K 9/6265; G06V 30/191; A61B 5/16; G06K 2009/6237

(57) ABSTRACT

A method of building a model for estimating a level of psychological safety includes acquiring, by a computer, post data communicated between members in a team, identifying fixed type post data that does not contribute to evaluation of the psychological safety among the acquired post data, and creating the model based on content of the acquired post data from which the fixed type post data has been removed.

### CROSS-REFERENCE TO RELATED APPLICATION

This application is based upon and claims the benefit of priority of the prior Japanese Patent Application No. 2021-41880, filed on Mar. 15, 2021, the entire contents of which are incorporated herein by reference.

### FIELD

The present embodiment relates to a method of building a model for estimating a level of psychological safety.

### BACKGROUND

In the field of office work such as research and development, work in charge of employees who make up a certain organization (team) is often complicatedly related to each other, and information sharing and cooperation between employees (users) is necessary for business execution. To realize smooth information sharing and cooperation within the team, it is important to maintain an environment in which the users may speak with peace of mind without feeling any risk or resistance. The degree to which a user may speak with peace of mind within a team is called psychological safety, and in recent years, it has been attracting attention from the viewpoint of corporate management and the like. A team manager needs to regularly check, for example, whether the psychological safety of the users in the team has deteriorated, so that the team in charge may be productive.

The psychological safety is defined as “a shared belief held by members of a team that the team is safe for interpersonal risk taking”.

As a method of obtaining the psychological safety, for example, the psychological safety may be obtained by conducting a questionnaire to the employees. Furthermore, there are the following techniques for obtaining the psychological safety by quantifying data. There is a technique of estimating a causal relationship between business data and supporting business improvement. In that technique, a non-linear term is added to an explanatory variable of business data and a multiple linear regression analysis is executed. An estimation model of an objective variable is calculated. The objective variable and the explanatory variable having a linear term are excluded from explanatory variable candidates as a same causal group. Furthermore, there is a technique of objectively evaluating a communication state of a team and supporting business execution by analyzing email transmission histories of team members. Furthermore, there is a technique of generating an employee problem prediction model from information such as emails, messages, and chats between employees, and predicting deterioration of relationships between employees and environmental problems. Furthermore, there is a technique of quantifying activeness of the team from the amount of communication in the chats. In that technique, text content of chats, senders/receivers of texts, and team information are input. A score is calculated in relation to communication of the team and team members such as connection between users and impact on others.

Japanese Laid-open Patent Publication No. 2018-156346; Japanese Laid-open Patent Publication No. 2004-220217; US Patent Publication No. 2019-0244152; Japanese Laid-open Patent Publication No. 2020-057067; and Amy Edmondson, “Psychological safety and learning behavior in work teams”, Administrative Science Quarterly, Vol. 44, No. 2 (Jun., 1999), pp. 350-383, Johnson Graduate School of Management, Cornell University are disclosed as related art.

### SUMMARY

According to an aspect of the embodiment, a method of building a model for estimating a level of psychological safety includes acquiring, by a computer, post data communicated between members in a team, identifying fixed type post data that does not contribute to evaluation of the psychological safety among the acquired post data, and creating the model based on content of the acquired post data from which the fixed type post data has been removed.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is a diagram illustrating an example of a model building method according to an embodiment;

FIG. 2 is a block diagram illustrating an exemplary functional configuration of an information processing device;

FIG. 3 is a diagram of an analysis state based on feature amounts;

FIG. 4 is a diagram of an analysis state based on feature amounts of an existing technique;

FIG. 5 is a diagram of an effect of fixed type posts on feature amounts by an existing technique;

FIG. 6 is a diagram of removal of the fixed type posts according to the embodiment;

FIG. 7 is a diagram of an effect of an interaction term of a feature amount;

FIG. 8 is a diagram of an effect of pruning;

FIG. 9 is a diagram of an exemplary questionnaire;

FIG. 10 is a diagram illustrating an exemplary hardware configuration of the information processing device;

FIG. 11 is a flowchart illustrating an example of model building processing according to the embodiment;

FIG. 12A is a flowchart illustrating a detailed example of fixed type post removal processing;

FIG. 12B is a flowchart illustrating a detailed example of post similarity determination processing;

FIG. 12C is a flowchart illustrating a detailed example of posting date and time periodicity determination processing;

FIG. 13 is a flowchart illustrating a detailed example of eature amount extraction processing for each team;

FIG. 14 is a block diagram illustrating another exemplary functional configuration of the information processing device;

FIG. 15 is a flowchart illustrating exemplary processing of outputting a psychological safety estimation result according to the embodiment; and

FIG. 16 is a diagram illustrating an exemplary display of an output result of a psychological safety estimation result.

### DESCRIPTION OF EMBODIMENT

The existing techniques have not been able to appropriately estimate the psychological safety of the user. For example, in the method using only a questionnaire, the burden increases for the user that answers the questionnaire every time the questionnaire is conducted on a continual basis.

Meanwhile, in the method of multiple linear regression analysis that builds an estimation model using data generated in a business process, all of extractable feature amounts are input, and the more types of explanatory variables, the larger the number of terms of the model to be output. Therefore, it becomes difficult to understand a result and utilize the result for improvement measures.

Furthermore, in the existing technique, in a case of analyzing chats, emails, and the like as the data generated in a business process, information other than text content and sender/receiver information is not considered. For example, in recent chats, non-text information such as reactions is used as a function to react to posting of text information, but the non-text information such as reactions is not able to be used as a feature amount at the time of analysis, and an analysis according to communication between users is not able to be performed.

As a result, in the existing techniques, a model for appropriately estimating the psychological safety of users has not been able to be built in the method of building a model from data generated in a business process. Furthermore, the existing techniques have not been able to appropriately present effective information for team management using the built model, for example, business improvement.

Hereinafter, an embodiment according to the disclosure will be described in detail with reference to the drawings.

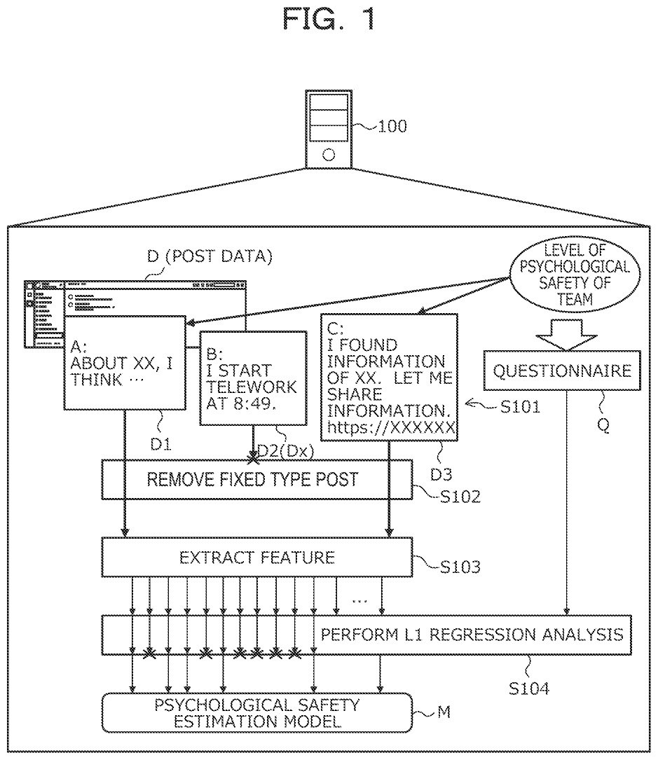

FIG. 1 is a diagram illustrating an example of a model building method according to an embodiment. In FIG. 1, an information processing device 100 is, for example, a computer device that creates (builds) a psychological safety estimation model of a team on the basis of business data communicated by members (users) in a team that performs predetermined work in a workplace. The information processing device 100 outputs information regarding determination of level of psychological safety using the created psychological safety estimation model.

The information processing device 100 inputs all pieces of information that may be acquired from the users in the team as information regarding psychological safety, and performs the following processing (1) to (3) to create an estimation model (psychological safety estimation model) M for each team.

(1) The information processing device 100 acquires the business data communicated in business by the users in the team (step S101). The business data is, for example, post data D such as chat data each communicated by the users in the team using a chat tool of a terminal. The information processing device 100 accesses a log database and acquires the post data D to be processed on the basis of condition settings such as a team name and a period of the post data D to be processed.

The user uses a chat tool provided in a smartphone or the like, and uses various functions such as channel, reaction, reply, and mention, in addition to send and receive using text information as the post data D. In a predetermined chat tool, communication between the users is performed within the channel.

The channels are created for different purposes such as by topic or by team. The user may join and leave the channel as needed. The reaction is a mechanism that responds to a post of a text sentence with a button such as a pictogram. In the chat, if the number of posts increases, past posts will flow to an outside of a display screen, so the user may easily and immediately react using the reaction. The reply is a mechanism that clearly indicates that a reply is to a certain post. The replay is used, for example, when a plurality of topics is being talked about in parallel on one channel, or when an old topic is mentioned later. The mention is a mechanism that clearly indicates who a post is addressed to by entering a user name in the post, and transmits a notification to a destination user at the same time.

In a case of performing an analysis using the psychological safety estimation model M, weighting (for example, a value as to which feature contributes to the estimation to what extent) of a feature amount corresponding to a function of a non-text sentence such as a reaction is unknown, and may vary from team to team. Therefore, in the present embodiment, an individual estimation model is created for each team.

In the reaction, a meaning such as “I understood” is imaged and replied as a reaction to a post. In recent chats, various functions such as reaction are added as non-text information, and the non-text information differs depending on the type of tool and may be changed in the future. Correspondingly, the information processing device 100 may identify the non-text information such as a reaction in the post data D and output text information corresponding to the non-text information. For example, the information processing device 100 recognizes a reaction image and converts a character string of the image into text information.

In addition, the information processing device 100 may hold text information given a meaning for each reaction in advance. For example, when a certain reaction is an image of “I understood”, the information processing device 100 stores identification information of the reaction and the text information of “I understood” in association with each other. Thereby, in a case where a certain reaction included in the acquired post data is the reaction of the image of “I understood”, the information processing device 100 may acquire the text information of “I understood” on the basis of the identification information of the reaction.

(2) The information processing device 100 removes the effect of the post data D that does not contribute to the evaluation of psychological safety among the acquired post data D (step S102). For example, the information processing device 100 determines the post data D corresponding to a fixed type post that occurs in business on the basis of the similarity and occurrence frequency of posts in units of contributors and posted places, and deletes the post data D of the fixed type post from data used to build the estimation model.

In the example of FIG. 1, among the post data D1, D2, and D3 of respective users A, B, and C, the post data D2 of the user B is a fixed type post Dx. The text information of the post data D2 “I start telework at 8:49” is a report that regularly occurs in teleworking work, and the information processing device 100 determines that this post data D2 (fixed type post Dx) is irrelevant to the psychological safety and excludes the post data from the analysis (x mark in the figure).

(3) The information processing device 100 extracts all the feature amounts that may be acquired from the post data D1 and D3 excluding the post data D2 of the fixed type post Dx removed in step S102 (step S103). The information processing device 100 extracts the feature amount for each user and the feature amount of the team as the feature amount. Although details will be described below, the feature amount for each user is the number of posts, the number of reactions, or the like, for example. The feature amount of the team is a feature amount calculated by the information processing device 100 for each team on the basis of the extracted feature amount for each user, and statistical amounts such as a mean, a variance, and a median of the number of posts and the number of reactions to a channel related to the users' team are calculated.

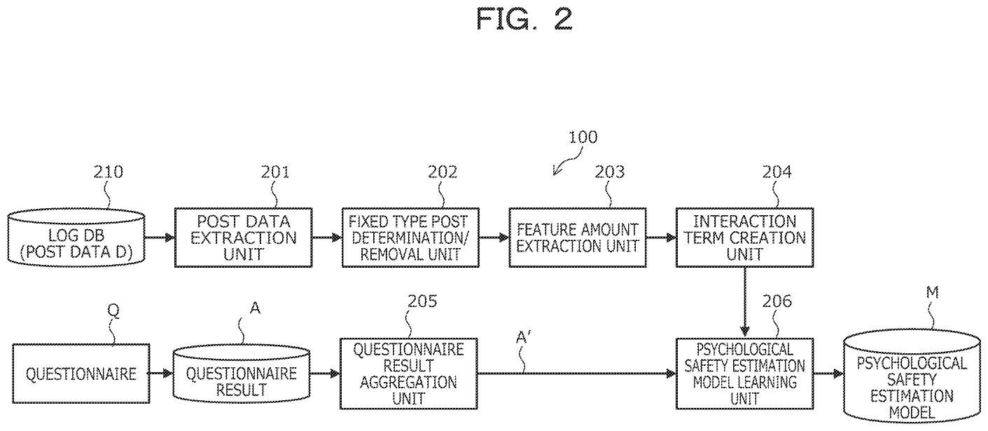

(4) The information processing device 100 performs an L1 regression analysis by L1 regularization (Lasso), using the feature amounts extracted in step S103 and a data aggregation value A′ (FIG. 2) of answers (questionnaire results) A (FIG. 2) to a questionnaire Q conducted for the users as explanatory variables (step S104).

As the questionnaire Q, the users are asked for questions about various items related to the evaluation of psychological safety in advance, and the data aggregation value A′ of the questionnaire result A by team and by time period is acquired.

The information processing device 100 adds an interaction term between feature amounts as an explanatory variable at the time of L1 regression analysis. In the present embodiment, the interaction term is included as an explanatory variable in order to capture the effect of the interaction, which is a synergistic effect that appears according to a combination of two feature amounts. The information processing device 100 improves the accuracy (prediction accuracy) of the L1 regression analysis by including the interaction term as an explanatory variable in pruning (corresponding to narrowing down output data) in the processing of L1 regression analysis.

The above interaction term will be briefly described. For example, in a case where the two feature amounts are “the number of posts and the number of characters”, the greater “the number of characters”, the higher the psychological safety tends to be, but the degree of effect of “the number of characters” is proportional to “the number of posts”. Therefore, “the number of posts and the number of characters” is used as an interaction term.

In step S104, the information processing device 100 omits unnecessary explanatory variables by the L1 regression analysis and creates the psychological safety estimation model M. In the L1 regression analysis of the present embodiment, pruning with a weight w set to 0 (zero) is performed for the unnecessary explanatory variables (x marks in the figure). As a result, the number of items to be referred to in the examination of improvement measures based on the psychological safety estimation model M may be reduced. In this respect, in an existing multivariate regression model, for example, multiple linear regression, all the feature amounts are input. Therefore, man-hours of an output estimation model increase, and utilization for improvement measures is difficult.

According to the information processing device 100 of the present embodiment, the estimation model may be created using only the posts that contribute to the evaluation of psychological safety by removing the fixed type posts that are irrelevant to the evaluation of psychological safety, and the accuracy of the estimation model may be improved. Furthermore, the information processing device 100 may improve the analysis accuracy and the accuracy of the estimation model by creating the estimation model in consideration of the interaction of the feature amounts of the posts that contribute to the evaluation of psychological safety.

Furthermore, even in the case where the post data includes an image (non-text information) such as a reaction, the information processing device 100 acquires the text information corresponding to the meaning of the reaction as data to be analyzed. Thereby, the non-text information included in the post data may be acquired without exception, and the estimation model that accurately reflects the communication between users may be created.

The information processing device 100 may present the created psychological safety estimation model M to the user who implements the improvement measures. The information processing device 100 displays a screen on which a psychological safety estimation value, explanatory variable values, objective variable prediction values, and the like of the psychological safety estimation model M are calculated. Thereby, the information processing device 100 may accurately and easily present detailed items that may cause changes in psychological safety to the user who implements the improvement measures.

FIG. 2 is a block diagram illustrating an exemplary functional configuration of the information processing device. The information processing device 100 includes a post data extraction unit 201, a fixed type post determination/removal unit 202, a feature amount extraction unit 203, an interaction term creation unit 204, a questionnaire result aggregation unit 205, and a psychological safety estimation model learning unit 206. Each of these functions may be obtained by executing a program by a computer (control unit) of the information processing device 100. The functions regarding creating the psychological safety estimation model on the basis of the post data and questionnaire aggregation will be described with reference to FIG. 2.

The post data extraction unit 201 acquires the post data to be analyzed. The post data extraction unit 201 acquires the post data to be analyzed corresponding the team members and the period to be analyzed set by input or the like from the log database 210 that accumulates and holds the post data D. In the case where the post data D includes the non-text information such as a reaction, the post data extraction unit 201 acquires the preset text information corresponding to the non-text information (reaction).

The fixed type post determination/removal unit 202 removes the post data of the fixed type post Dx from the acquired post data D on the basis of the similarity of the post data D, the periodicity of the posting date and time, and the like. The feature amount extraction unit 203 extracts all the feature amounts that may be acquired from the post data D excluding the fixed type post Dx. For example, the feature amount for each user is the number of posts, the number of reactions, or the like, for example. The feature amount of the team is a feature amount calculated by the information processing device 100 for each team on the basis of the extracted feature amount for each user, and the information processing device 100 calculates statistical amounts such as a mean, a variance, and a median of the number of posts and the number of reactions to a channel related to the users' team.

The interaction term creation unit 204 adds, as an explanatory variable, the interaction term between the feature amounts included in the post data D after the fixed type post Dx is removed by the fixed type post determination/removal unit 202. The questionnaire result aggregation unit 205 acquires the questionnaire result A of the questionnaire Q regarding psychological safety conducted in advance for the users of the team of the post data, and obtains the data aggregation value A′ of the questionnaire result A by team and by time period.

The psychological safety estimation model learning unit 206 creates the psychological safety estimation model M by performing a regression analysis by L1 regularization (L1 regression analysis), using the feature amounts including the interaction term created by the interaction term creation unit 204 and the data aggregation value A′ of the questionnaire result A as explanatory variables.

### Comparison Between Embodiment and Existing Technique

Here, a comparison between the processing according to the embodiment and processing according to an existing technique will be described.

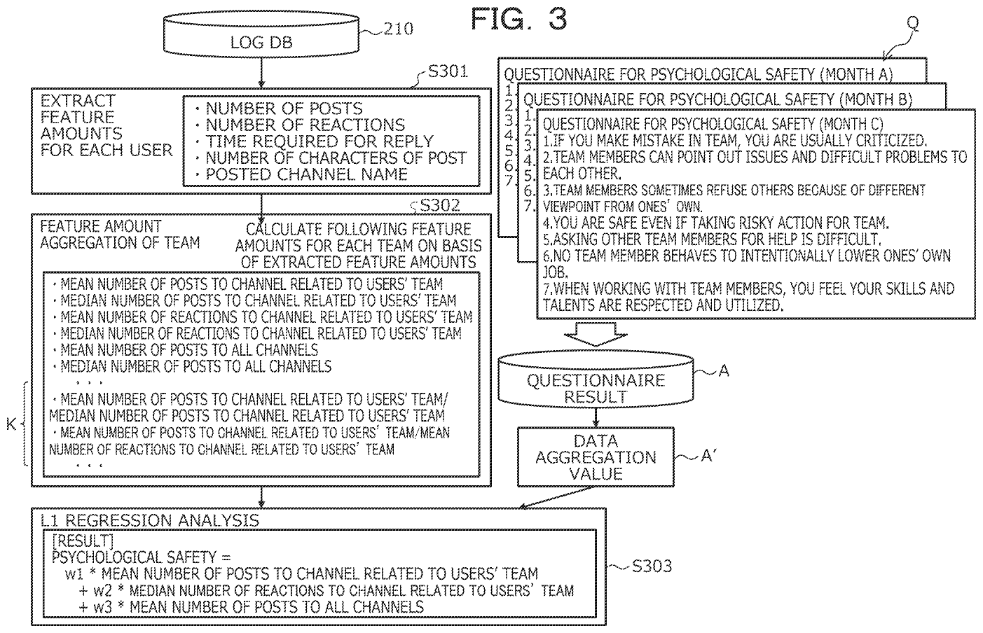

FIG. 3 is a diagram of an analysis state based on feature amounts. FIG. 3 mainly illustrates a processing state of the feature amounts by the feature amount extraction unit 203, the interaction term creation unit 204, and the psychological safety estimation model learning unit 206 of FIG. 2.

The feature amount extraction unit 203 extracts the feature amounts for each user that may be acquired from the post data D accumulated and held in the log database 210 (step S301). The feature amounts are, for example, the number of posts, the number of reactions, the time required for reply (time after posting to reply), the number of characters in a post, the number of posts in a team channel, and the like.

The feature amount extraction unit 203 calculates statistical amounts such as the mean, variance, median, and the like of the acquired data (step S302). The feature amount extraction unit 203 calculates the feature amounts for each team on the basis of the feature amounts extracted in step S301. For example, as illustrated in FIG. 3, “a mean number of posts to the channel related to the users' team”, “a median number of posts to the channel related to the users' team”, “a median number of posts to all of channels”, and the like are calculated.

The interaction term creation unit 204 calculates the interaction term of a combination of a plurality of calculated feature amounts. For example, as illustrated in FIG. 3, the interaction term such as “the mean number of posts to the channel related to the users' team/the median number of posts to the channel related to the users' team”, or the like is calculated (the reference code K in FIG. 3).

The psychological safety estimation model learning unit 206 performs the L1 regression analysis, using the feature amounts extracted in step S302 and the data aggregation value A′ of the questionnaire Q conducted in advance as explanatory variables (step S303). The psychological safety estimation model M is created by the L1 regression analysis.

Regarding the questionnaire Q, for example, the questionnaire Q including a plurality of question items for psychological safety is conducted monthly for the users of the team. The data aggregation value A′ for each team and for each time (monthly, or the like) of the questionnaire result A answered by the users is input to the psychological safety estimation model learning unit 206. In the example of FIG. 3, seven question items are included, and the psychological safety estimation model learning unit 206 estimates the level of psychological safety for the seven question items (a prediction value Y of the psychological safety, a calculation equation will be described below) by the L1 regression analysis.

In the L1 regression analysis performed by the psychological safety estimation model learning unit 206, the number of items of the model to be output may be reduced by pruning the unnecessary explanatory variables with the weight w set to 0 (zero). FIG. 3 illustrates an example in which the psychological safety estimation model M has three terms.

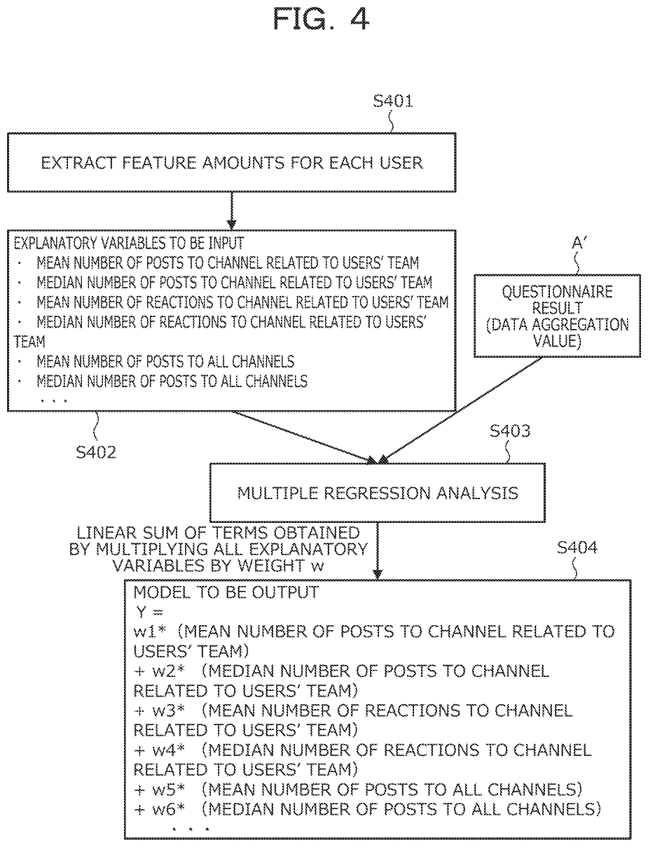

FIG. 4 is a diagram of an analysis state based on feature amounts of an existing technique. FIG. 4 is illustrated for comparison with FIG. 3. In the existing technique, the acquirable feature amounts for each user are extracted (step S401). Then, the explanatory variables to be input to the regression analysis are calculated (step S402), and the multiple regression analysis is performed using the calculated explanatory variables and the data aggregation value A′ of the questionnaire result A (step S403).

In this multiple regression analysis, a linear sum of terms obtained by multiplying all the explanatory variables by the weight w is calculated. Therefore, in the existing technique, the number of items of the psychological safety estimation model to be output becomes large. For example, if the number of explanatory variables extracted in step S402 is 17,442, the number of items of the psychological safety estimation model to be output in step S404 is 17,442, which is a large number.

By the way, when the number of feature amounts calculated in step S302 is 17,442 in the present embodiment described with reference to FIG. 3, the number of items of the psychological safety estimation model to be output is narrowed down to three by the above pruning performed in step S303.

In addition, the existing technique does not consider (calculate) the interaction term in step S402. If the interaction term is calculated in step S402, the number of items of the psychological safety estimation model increases by the number of the calculated interaction terms in step S404.

In this way, according to the embodiment, the number of items of the psychological safety estimation model M to be output is narrowed down, and the number of items presented to the user may be reduced, so that the improvement measures may be accurately taken.

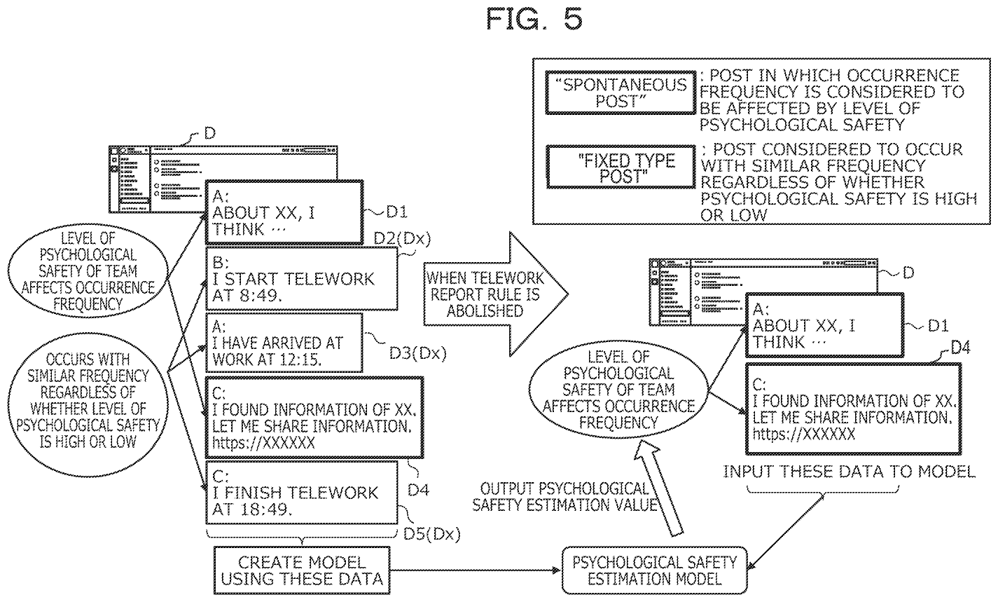

FIG. 5 is a diagram of an effect of the fixed type posts on the feature amounts by the existing technique. Problems in the case where post data D includes the fixed type post Dx when the processing illustrated in FIG. 4 is performed by the existing technique will be described.

In chats between users, communication that does not contribute to the evaluation of psychological safety may occur. For example, there is a fixed type post such as the above telework report. This fixed type post is unnecessary data for estimating psychological safety, but in the existing technique, the fixed type post is used as a feature amount of input of the model.

For example, among the post data D on the left side of FIG. 5, the occurrence frequency for a “spontaneous post” by the user is affected by the level of psychological safety of the team. The “spontaneous post” is, for example, a post whose occurrence frequency is considered to be affected by the level of psychological safety, and corresponds to the post data D1 of the user A “About XX, I think . . . ”, and the post data D4 of the user C.

However, the post data D includes the fixed type post Dx that occurs with a similar frequency regardless of whether the psychological safety is high or low. In the example on the left side of FIG. 5, the post data D2, D3, and D5 are the fixed type post Dx of the telework report

Here, a state in a case where the rule of telework report itself is abolished is illustrated on the right side of FIG. 5. In business, in the case where the telework report by each user is simply abolished, the number of “spontaneous posts” that is originally desired to observe is the same as the number on the left side of FIG. 5, but the total number of posts will decrease as the fixed type post Dx disappears.

Here, in the present embodiment, the rule of telework report is not abolished, and the information processing device 100 (post data extraction unit 201) acquires the posts including the fixed type post Dx from the post log database. As described above, the post data extraction unit 201 performs the processing of acquiring the post data including the fixed type post Dx from the log database, and then excluding the fixed type post Dx from the data to be analyzed, as unnecessary data for the L1 regression analysis to be processed thereafter.

As a method of removing a fixed phrase such as the fixed type post Dx, there is a method using keyword determination. However, the content of the fixed type post Dx (text content) may differ in expression depending on an individual or a scene, and the fixed type post. Dx is not able to be correctly removed only by keyword determination.

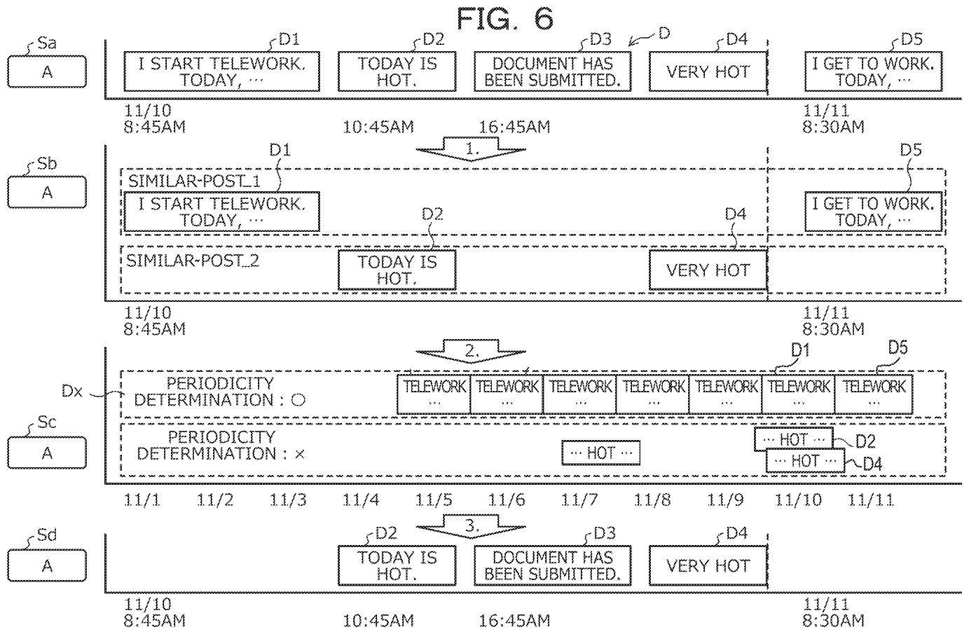

FIG. 6 is a diagram of removal of the fixed type posts according to the embodiment. An example of excluding the fixed type post Dx from the post data D of the user A in the information processing device 100 (post data extraction unit 201) is illustrated. Sa to Sd in FIG. 6 illustrated on the vertical axis are states for each processing for the post data D, and the horizontal axis represents the time (posting time).

The post data extraction unit 201 identifies similar posts for the plurality of post data D of the user A by the following processing 1 to 3.

1. The post data extraction unit 201 extracts a set of posts having high similarity in the content (text information) of the posts by the same contributor or the same channel of a chat tool.

2. The post data extraction unit 201 acquires the posting date and time of the extracted set of posts. For example, whether posts occur at a continuous frequency on or after a specific day is determined.

3. The post data extraction unit 201 identifies the post data that occurs at a continuous frequency as the fixed type post Dx, and excludes the post data from the data used for the L1 regression analysis.

In the example illustrated in FIG. 6, first, as illustrated in Sa, the user A posts each post data D on the chat. Then, as illustrated in Sb, the post data extraction unit 201 classifies each post data D into a similar-post_1 or a similar-post_2 on the basis of the similarity of sentences. For example, the post data extraction unit 201 determines that the post data D1 and D5 are both the similar-post_1 on the basis of the wording “I start”. Furthermore, the post data extraction unit 201 determines that the post data D2 and D4 are both the similar-post_2 on the basis of the wording “hot”.

Furthermore, as illustrated in Sc, the post data extraction unit 201 classifies the post data D, which has been classified according to the similarity, into periodicity (○ mark) or non-periodicity (x mark) on the basis of the periodicity of the post. For example, the post data extraction unit 201 determines that the post data D1, D5, and the like with the posting time of the post data D that falls within a predetermined time range (8:30 to 8:45 AM in the illustrated example) has periodicity (○). Furthermore, the post data extraction unit 201 determines that, among the post data D, the post data D2, D4, and the like having no periodicity in the posting time as non-periodicity (x).

Then, as illustrated in Sd, the post data extraction unit 201 identifies the post data D (D1, D5, and the like) determined to have periodicity (○) as the fixed type post Dx, and removes the fixed type post Dx. Thereby, the post data D (D2, D3, and D4) determined to have no periodicity (x) remain and are used as data (feature amounts) for the L1 regression analysis.

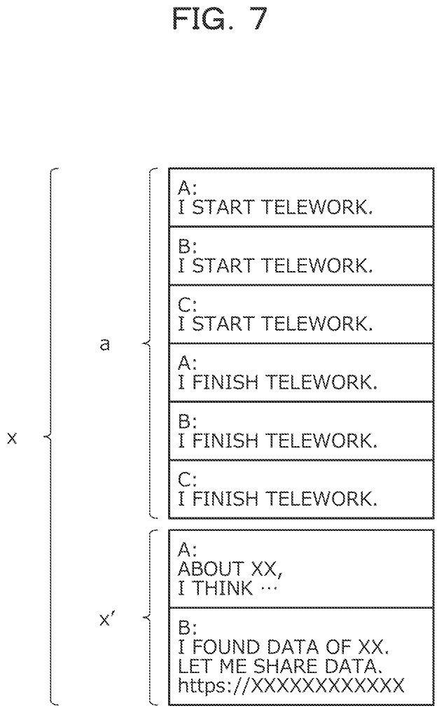

FIG. 7 is a diagram of an effect of an interaction term of a feature amount. Here, x is a feature amount (the number of posts, the number of reactions, or the like), and Y is a prediction value of psychological safety. In the absence of an interaction term, an offset effect is considered to be removed by a constant term. However, in a case where the model includes an interaction term, it is difficult to remove the offset effect from the prediction value Y of psychological safety for the following reasons.

In a case where x contains an offset, x′ and a are defined as follows and x=x′+a is set (a: an offset, x′: a (true) feature amount excluding the offset).

(1) In the case of only a single feature amount term

$$Y = w 1 * x 1 + … + b = w 1 * ( x {1}^{′} + a 1 ) + … + b = w 1 * x {1}^{′} + w 1 * a 1 + … + b$$

(2) In the case of including an interaction term

$$Y = w * x 1 * x 2 + … + b = w * ( {x }^{′} 1 + a 1 ) ( {x}^{′} 2 + a 2 ) + … + b = w * {x}^{′} 1 * {x}^{′} 2 + w * a 1 * {x}^{′} 2 + w * a 2 * {x}^{′} 1 + w * a 1 * a 2 + … + b$$

As described above, in the case of including an interaction term, a term in which x′ and a are mixed is generated, and thus removal becomes difficult. That is, since a (offset) to be removed is multiplied by the variable x′, the offset cannot be completely removed with the constant term b alone.

In this regard, in the present embodiment, the L1 regression analysis is performed after removing the fixed type post Dx corresponding to the offset a. Thereby, the above offset effect may be removed, and the psychological safety may be estimated using the post data D that is originally desired to be observed as a feature amount. Furthermore, the interaction term calculated on the basis of the feature amount after the fixed type post Dx is removed may be used for the analysis.

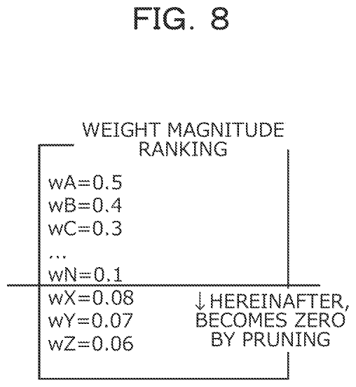

FIG. 8 is a diagram of an effect of pruning. Problems in a case of simply performing pruning in a regression analysis will be explained. Here, it is assumed that the following model is created as the psychological safety estimation model.

Y=wX*(x1+a1)+w2*x2+w3*x3+w4*x4+ . . . +b

In the above model, by adding a1 (offset), the weight wX of that term becomes smaller and the degree of contribution decreases. If pruning (for example, removal of the weight of 0.1 or less) is performed in this state, a term with an offset tends to be unreasonably pruned as illustrated in FIG. 8.

In this regard, in the present embodiment, the L1 regression analysis is performed after removing the fixed type post (fixed type post Dx) corresponding to the offset al. Thereby, appropriate pruning may be performed without including unnecessary a1 (offset) itself.

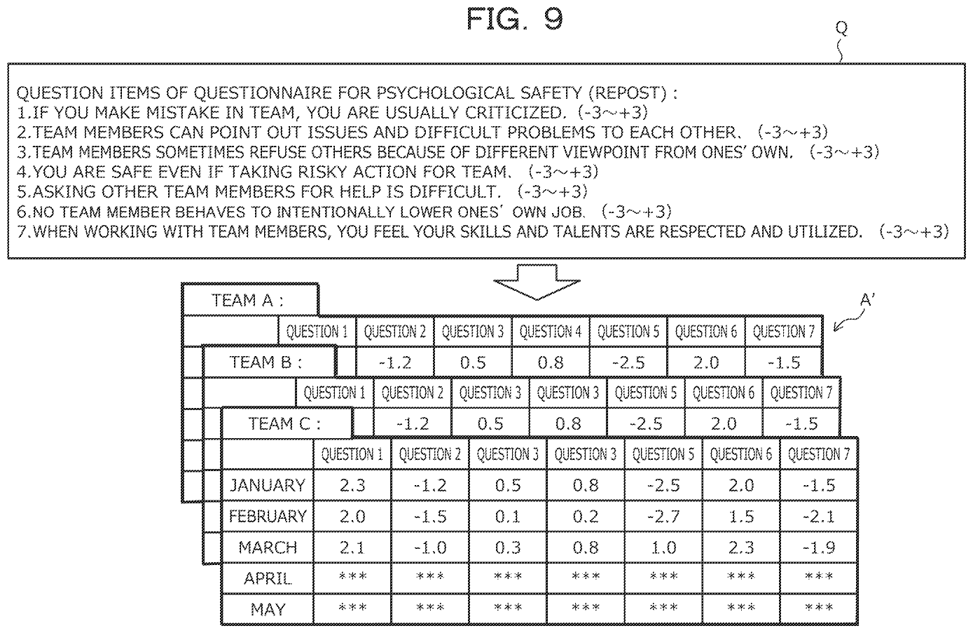

FIG. 9 is a diagram of an exemplary questionnaire. When creating the psychological safety estimation model, the information processing device 100 performs the L1 regression analysis, using the feature amounts of the post data D and the data aggregation value A′ of the questionnaire result A for the questionnaire Q conducted for the users.

The content of the questionnaire Q illustrated on the upper part of FIG. 9 includes each of question items similar to those in FIG. 3. The question items are, for example, “1. If you make a mistake in the team, you are usually criticized.”, “2. Team members can point out issues and difficult problems to each other.”, . . . , “7. When working with team members, you feel that your skills and talents are respected and utilized.”, and the like.

For each of these question items, the team members (users) answer a numerical value from +3 to −3 centered on 0. +/− is the degree of consent of the members to the question item, the larger the value of + (plus), the higher the degree of consent to the question item, and the larger the value of − (minus), the lower the degree of consent to the question item.

The lower part of FIG. 9 illustrates an example of the aggregation value A′ of the questionnaire result A. The information processing device 100 (questionnaire result aggregation unit 205) calculates the aggregation value A′ for each team for each period (monthly) on the basis of the questionnaire result A. In the questionnaire result aggregation unit 205, for example, for the team C illustrated in the lower part of FIG. 9, the vertical axis represents each period (each month) and the horizontal axis represents each of the questions 1 to 7, and the mean of the questionnaire result A (value +3 to −3) answered by each member in the team is stored as the aggregation value A′ in an intersecting square part. The questionnaire result aggregation unit 205 outputs the aggregation value A′ for each team for each period illustrated in the lower part of FIG. 9 to the psychological safety estimation model learning unit 206.

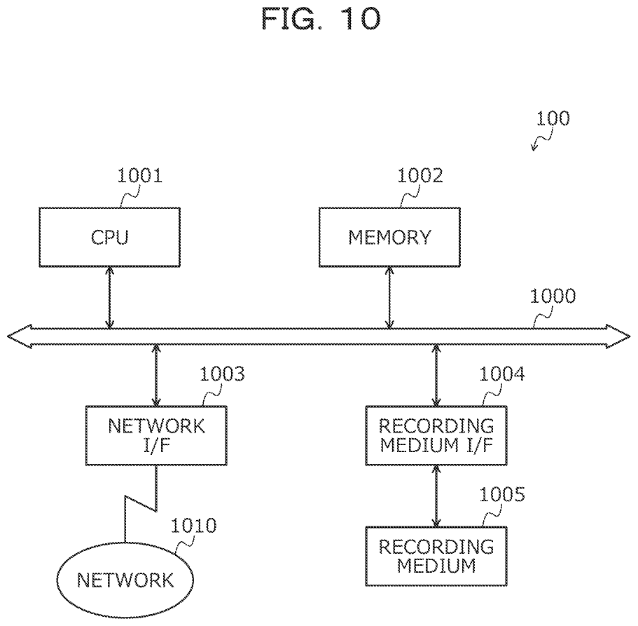

FIG. 10 is a diagram illustrating an exemplary hardware configuration of the information processing device. The information processing device 100 may be configured by a computer such as a server including general-purpose hardware illustrated in FIG. 10.

The information processing device 100 includes a central processing unit (CPU) 1001, a memory 1002, a network interface (IF) 1003, a recording medium IF 1004, and a recording medium 1005. A bus 1000 connects each of the units.

The CPU 1001 is an arithmetic processing unit that functions as a control unit that controls the entire information processing device 100. The memory 1002 includes a nonvolatile memory and a volatile memory. The nonvolatile memory is, for example, a read only memory (ROM) that stores a program of the CPU 1001. The volatile memory is, for example, a dynamic random access memory (DRAM) or a static random access memory (SRAM) used as a work area of the CPU 1001.

The network IF 1003 is a communication interface for a network 1010 such as a local area network (LAN), a wide area network (WAN), or the Internet. The information processing device 100 communicates with the network 1010 via the network IF 1003. For example, the information processing device 100 communicates with a device outside, for example, a log DB 210 in FIG. 2, via the network 1010, and acquires log data.

The recording medium IF 1004 is an interface for writing and reading information processed by the CPU 1001 to and from the recording medium 1005. The recording medium 1005 is a recording device that assists the memory 1002. As a recording device, a hard disk drive (HDD), a solid state drive (SSD), a universal serial bus (USB) flash drive, or the like may be used.

When the CPU 1001 executes the program recorded in the memory 1002 or the recording medium 1005, each function as the control unit of the information processing device 100, for example, the post data extraction unit 201 to the psychological safety estimation model learning unit 206 in FIG. 2, is implemented. The memory 1002 and the recording medium 1005 stores a database of the information processing device 100, for example, the data of the questionnaire result A illustrated in FIG. 2 and the data of the psychological safety estimation model M.

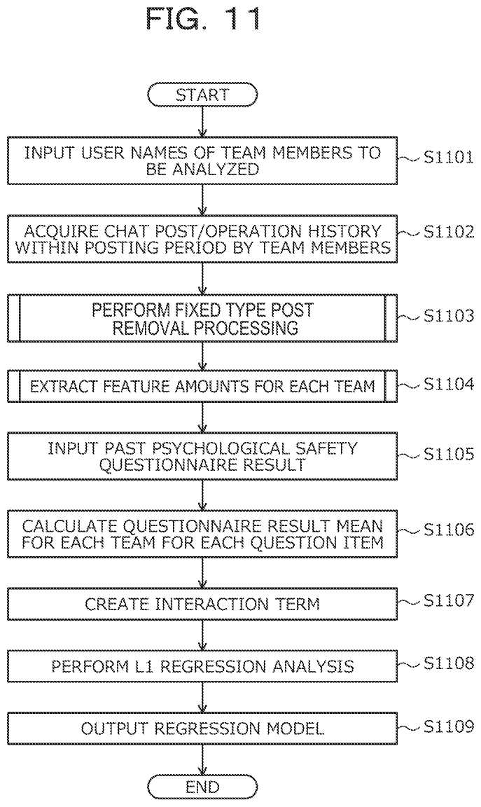

FIG. 11 is a flowchart illustrating an example of model building processing according to the embodiment. Exemplary processing of creating a model (psychological safety estimation model M) performed by the control unit (CPU1001) of the information processing device 100 will be described in order.

First, conditions for creating a model are set by the user for the information processing device 100. As a condition, user names of the team members to be analyzed are input to the information processing device 100 (step S1101).

Then, the information processing device 100 acquires chat post/operation history by the team members within a posting period (step S1102). The information processing device 100 acquires the post data D of the team members for a predetermined period from the log database 210. The operation history corresponds to, for example, an attribute of the post data D, and includes operations such as the reaction, reply, mention, and the like described above. Furthermore, the information processing device 100 converts the non-text information included in the acquired post data D into text information.

Next, the information processing device 100 removes the fixed type post Dx included in the acquired post data D (step S1103).

Next, the information processing device 100 extracts the feature amounts for each team, using the content (text information) of the post data D from which the fixed type posts have been removed as the feature amounts (step S1104).

The information processing device 100 receives the input of the questionnaire result A of the questionnaire Q regarding past psychological safety conducted for the users (step S1105). Then, the information processing device 100 calculates the mean of the questionnaire result A for each team for each question item for the input questionnaire result A (step S1106).

Thereafter, the information processing device 100 creates the interaction term of the feature amounts (step S1107). Then, the information processing device 100 performs the L1 regression analysis for the feature amounts including the interaction term (step S1108). Thereby, the information processing device 100 creates the regression model (psychological safety estimation model M) and outputs the model to the outside (step S1109). A series of model creation processing ends.

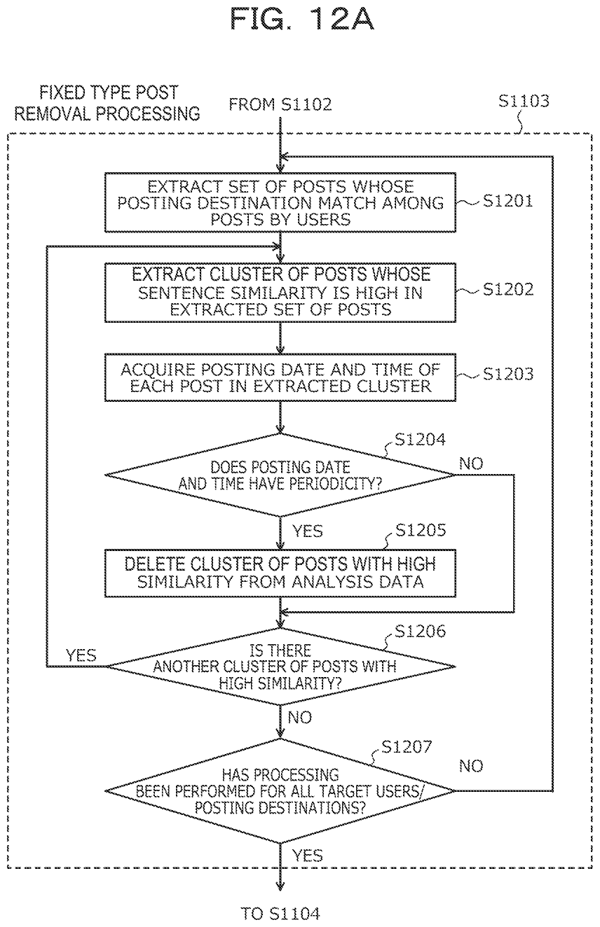

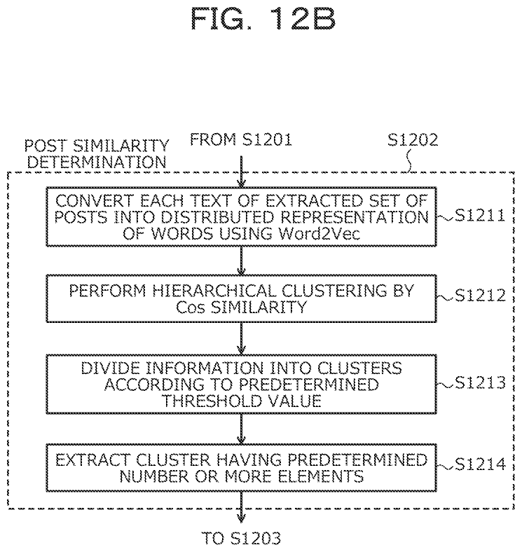

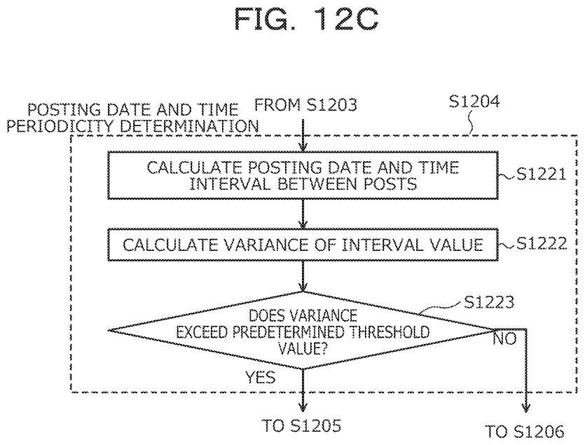

FIG. 12A is a flowchart illustrating a detailed example of fixed type post removal processing. The processing illustrated in FIG. 12A is a sub flow that details the processing of step S1103 of FIG. 11, and is processing corresponding to the diagram of FIG. 6.

In the fixed type post removal processing, the information processing device 100 extracts a set of posts (post data D) whose posting destinations match among the posts by each of the users (step S1201). Next, the information processing device 100 extracts a cluster (set) of posts having high sentence similarity from the extracted set of posts (step S1202). Next, the information processing device 100 acquires the posting date and time of each post (post data D) in the extracted cluster (step S1203).

Next, the information processing device 100 determines whether the posting date and time of the post data D has periodicity (step S1204). As a result of the determination, when the posting date and time have periodicity (step S1204: Yes), the information processing device 100 deletes the cluster of posts having high similarity (post data D) from the analysis data (step S1205) and proceeds to the processing of step S1206. On the other hand, as a result of the determination, when the posting date and time has no periodicity (step S1204: No), the information processing device 100 proceeds to the processing of step S1206.

In step S1206, the information processing device 100 determines whether there is another cluster of posts having high similarity (step S1206). As a result of the determination, when there is another cluster of posts having high similarity (step S1206: Yes), the information processing device 100 returns to the processing of step S1202. On the other hand, as a result of the determination, when there is no other cluster of posts having high similarity (step S1206: No), the information processing device 100 determines whether the above-described processing has been performed for all the target users/posting destinations (step S1207).

As a result of the determination in step S1207, when the processing is not performed for some target user/posting destination (step S1207: No), the information processing device 100 returns to the processing of step S1201. On the other hand, when the processing has been performed for all the target users/posting destinations (step S1207: Yes), the information processing device 100 terminates the above fixed type post removal processing and proceeds to the processing of step S1104 (see FIG. 11).

FIG. 12B is a flowchart illustrating a detailed example of post similarity determination processing. The processing illustrated in FIG. 12B is a sub flow that details the processing of step S1202 in FIG. 12A.

In post similarity determination processing, the information processing device 100 converts the text (text information) of each set of posts extracted in step S1201 (FIG. 12A) into a distributed representation of words (step S1211). For example, the information processing device 100 converts the text information of each set of posts into a distributed representation of words by using a method such as Word2Vec.

Next, the information processing device 100 hierarchically clusters the text information by similarity by using the Cos similarity method or the like (step S1212), and then divides the hierarchically clustered text information into clusters according to a predetermined threshold value (step S1213).

After that, the information processing device 100 extracts the cluster having a predetermined number of elements or more (step S1214). Then, the information processing device 100 terminates the above post similarity determination processing and proceeds to the processing of step S1203 (FIG. 12A).

FIG. 12C is a flowchart illustrating a detailed example of posting date and time periodicity determination processing. The processing illustrated in FIG. 12C is a sub flow that details the processing of step S1204 in FIG. 12A.

In the posting date and time periodicity determination processing, the information processing device 100 calculates a posting date and time interval between the posts in the cluster acquired in step S1203 (FIG. 12A) (step S1221). Next, the information processing device 100 calculates a variance of the calculated interval value of the posting date and time (step S1222). Then, the information processing device 100 determines whether the calculated variance exceeds a predetermined threshold value.

As a result of the determination, when the calculated variance exceeds the predetermined threshold value (step S1223: Yes), the processing proceeds to step S1205 (FIG. 12A). On the other hand, when the calculated variance does not exceed the predetermined threshold value (step S1223: No), the processing proceeds to step S1206 (FIG. 12A). Then, the information processing device 100 terminates the posting date and time periodicity determination processing.

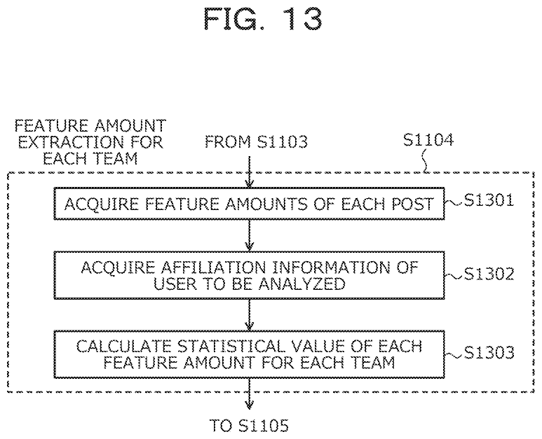

FIG. 13 is a flowchart illustrating a detailed example of feature amount extraction processing for each team. The processing illustrated in FIG. 13 is a sub flow that details the processing of step S1104 in FIG. 11.

In the feature amount extraction processing for each team, the information processing device 100 acquires the feature amount of each post (post data D) after the fixed type post removal processing in step S1103 (see FIGS. 11 and 12A) (step S1301).

Next, the information processing device 100 acquires affiliation information of the user to be analyzed (step S1302). The affiliation information is the team name to which the user belongs or the like. Next, the information processing device 100 calculates the statistical value of each feature amount for each team (step S1303). For example, as illustrated in FIG. 3, the statistical values are, for example, “a mean number of posts to the channel related to the users' team”, “a median number of posts to the channel related to the users' team”, “a median number of posts to all of channels”, and the like. Then, the information processing device 100 terminates the feature amount extraction processing for each team.

### Example of Presentation of Psychological Safety Estimation Result Using Estimation Model

Next, an example of presenting the estimation result of psychological safety by using the psychological safety estimation model M created above will be described.

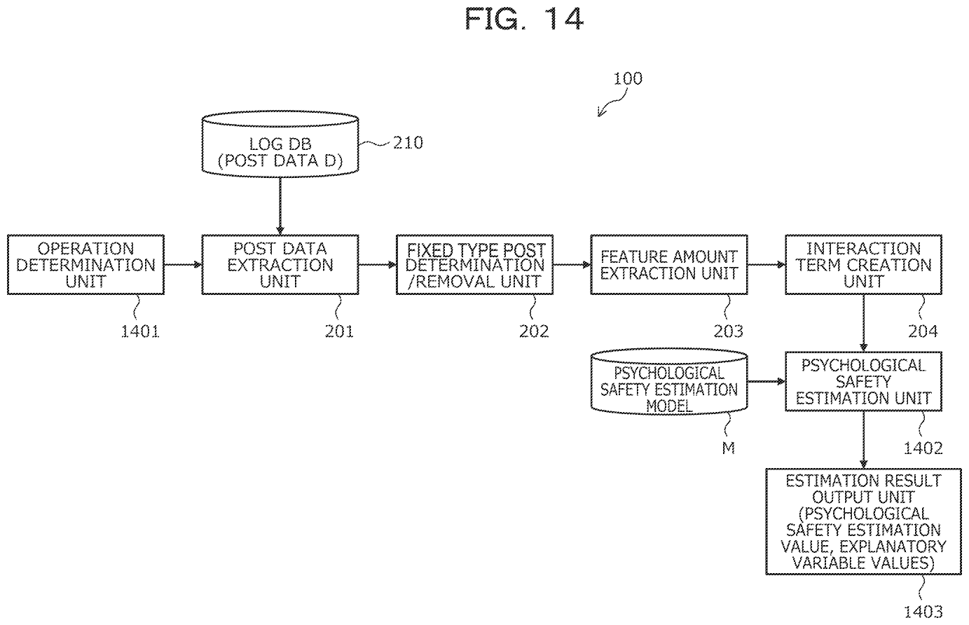

FIG. 14 is a block diagram illustrating another exemplary functional configuration of the information processing device. In FIG. 14, similar functional units to those in FIG. 2 are given the same reference numerals. The information processing device 100 has a function to present information useful for grasping psychological safety and improvement measures by using the created psychological safety estimation model M. Note that the information processing device 100 may have each of the functional units illustrated in FIG. 2 and each of the functional units illustrated in FIG. 14.

The information processing device 100 illustrated in FIG. 14 includes an operation determination unit 1401, a psychological safety estimation unit 1402, and an estimation result output unit 1403, in addition to each of the functional units described in FIG. 2. The operation determination unit 1401 sets operation date and time (business day), period, and the like of the team to be analyzed among the posts (post data D), as extraction conditions for acquiring post data by the post data extraction unit 201.

The psychological safety estimation unit 1402 refers to the created psychological safety estimation model M and calculates various estimation results related to psychological safety as concrete numerical values. For example, a prediction value of the objective variable, the explanatory variable, and the like are calculated. The estimation result output unit 1403 presents the various estimation results related to psychological safety on the display screen or the like to the user who grasps the psychological safety and takes improvement measures.

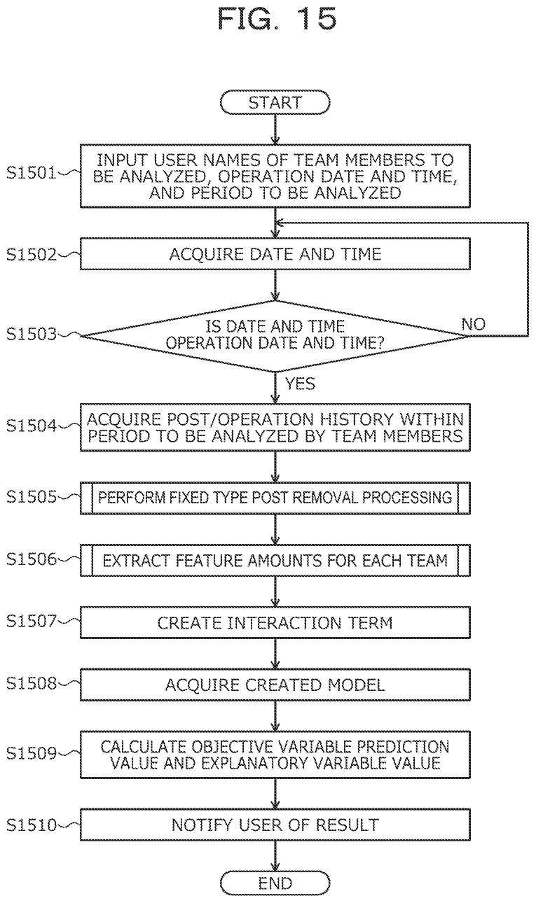

FIG. 15 is a flowchart illustrating exemplary processing of outputting a psychological safety estimation result according to the embodiment. Exemplary processing of outputting various estimation results related to psychological safety by referring to the psychological safety estimation model M performed by functions of the information processing device 100 illustrated in FIG. 14 will be described.

First, the user names of the team members to be analyzed, the operation date and time, and the period to be analyzed are input to the information processing device 100 (step S1501).

Then, the information processing device 100 acquires the input date and time (step S1502) and determines whether the acquired date and time is the operation date and time (step S1503). In the case where the acquired date and time is the operation date and time (step S1503: Yes), the information processing device 100 acquires the posts (post data D) within the period to be analyzed and the operation history of the corresponding team members from the log database 210 (step S1504). In the case where the acquired date and time is not the operation date and time (step S1503: No), the information processing device 100 returns to the processing of step S1502.

Next, the information processing device 100 removes the fixed type post Dx included in the acquired post data D (step S1505). Details of the processing in step S1505 are similar to those in FIG. 12A.

Next, the information processing device 100 extracts the feature amounts for each team, using the content (text information) of the post data D from which the fixed type posts have been removed as the feature amounts (step S1506). Details of the processing in step S1506 are similar to those in FIG. 13.

Thereafter, the information processing device 100 creates the interaction term of the feature amounts (step S1507). Then, the information processing device 100 acquires the created model (psychological safety estimation model M) (step S1508).

Then, the information processing device 100 refers to the psychological safety estimation model M, and calculates the prediction value of the objective variable and the value of the explanatory variable on the basis of the calculated feature amounts including the interaction term (step S1509). The information processing device 100 notifies the user outside of the calculated values and the like (step S1510) and terminates the series of processing.

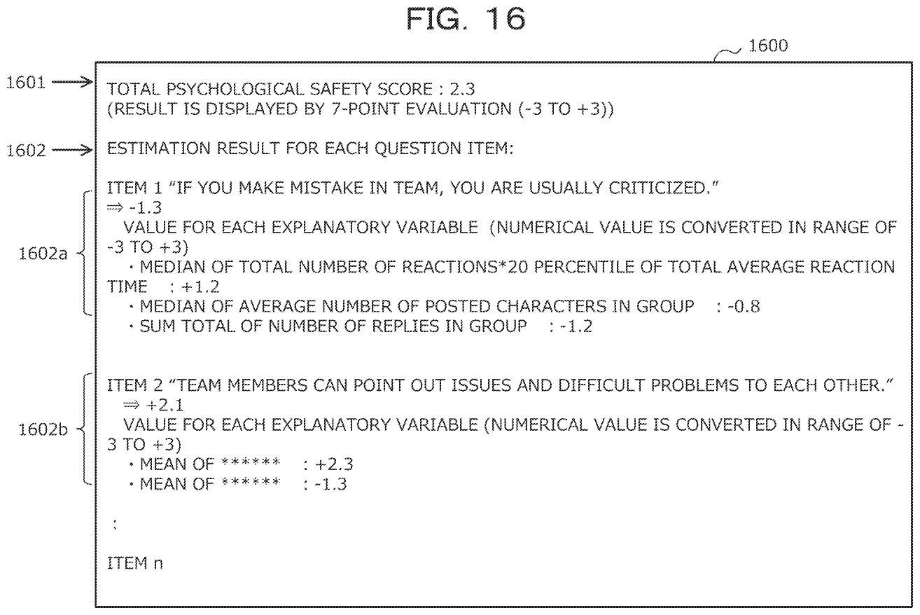

FIG. 16 is a diagram illustrating an exemplary display of an output result of a psychological safety estimation result. The information processing device 100 displays, for example, each information of the psychological safety estimation result on a display screen 1600 illustrated in FIG. 16.

The information processing device 100 displays information regarding a total psychological safety score 1601 and an estimation result 1602 for each question item on the display screen 1600, for example. The total psychological safety score 1601 is a numerical value of the psychological safety of the corresponding team, and is displayed as a score for a 7-point evaluation with a value of −3 to +3. This total psychological safety score 1601 is, for example, the mean of each of the scores of the estimation result 1502 for each question item.

The information processing device 100 displays the estimation results 1602a, 1602b, and the like for each questionnaire item conducted in the questionnaire Q, in the estimation result 1602 for each question item. In the example of FIG. 16, in an estimation result 1602a of item 1, the score for the questionnaire Q that “If you make a mistake in the team, you are usually criticized.” is “−1.3”, and the value for each explanatory variable is displayed. For example, the value “+1.2” of the explanatory variable “the median of the total number of reactions*20 percentile of the total average reaction time” is displayed.

From the various estimation results related to psychological safety displayed in FIG. 16, the user such as a team manager may specifically grasp the psychological safety of the team by the display of the values for each item. Furthermore, since details such as which item is the bottleneck may be grasped, improvement measures may be appropriately implemented.

In the above embodiment, an example in which the information processing device 100 creates the psychological safety estimation model on the basis of the post data generated in business has been described, but the present embodiment is not limited to the example. The psychological safety estimation model may be applied not only to work but also to various data communicated by a plurality of users (members) within a predetermined team to obtain similar functions and effects. Furthermore, the information processing device 100 may create the psychological safety estimation model including not only the post data such as chats but also email, data after conversation recognition, and the like.

According to the above-described embodiment, the information processing device 100 builds the model for estimating the level of psychological safety in the team. The information processing device 100 acquires the post data communicated by the members in the team. The information processing device 100 identifies, from among the post data, the fixed type post data that does not contribute to the evaluation of psychological safety, and creates the model on the basis of the content of the post data from which the fixed type post data has been removed. As a result, a model based on the post data that contributes to psychological safety may be built, and the accuracy of the model may be improved.

The information processing device 100 identifies the fixed type post data on the basis of the similarity of the content of the post in units of the contributor of the post data or in units of the tool used for the post, and the occurrence frequency of the post data. As a result, it becomes possible to exclude the post data such as regular business reports from the large number of post data and build a model with good accuracy.

To create the model, the information processing device 100 extracts the feature amounts from the content of the post data for each member, calculates the statistical values of the feature amounts for each team, and performs the regression analysis by L1 regularization. Thereby, the unnecessary explanatory variables may be pruned by the L1 regression analysis after extracting the feature amounts from the content of the post data excluding the fixed type post data. Thereby, the number of items output by the built model may be reduced to only effective ones, and improvement measures may be appropriately implemented.

The information processing device 100 calculates the statistical values of the feature amounts for each team including the interaction term of a combination of two different feature amounts. Thereby, the interaction term of a combination of two different feature amounts is added as an explanatory variable, and then pruning is performed by the L1 regression analysis. Therefore, estimation may be performed including the synergistic effect exerted due to the interaction term, and the estimation accuracy may be further improved.

The information processing device 100 performs the regression analysis including the input of the data aggregation value of the questionnaire result of the question items regarding the estimation of psychological safety conducted in advance for the members. Thereby, the psychological safety of each question item in the questionnaire may be concretely presented as a numerical value.

The information processing device 100 may acquire the post data by converting the non-text information included in the post data into text and extracting the text. Thereby, the non-text information included in the post data on chats may also be acquired as the feature amounts, and the psychological safety estimation model that reflects the communication by chats between the members may be built.

The information processing device 100 refers to the model created on the basis of the post data corresponding to the setting input of the predetermined team and the period to be analyzed and outputs information regarding the psychological safety of the team during the period. Thereby, the information regarding psychological safety of the team may be accurately presented by referring to the built model, and the improvement measures may be appropriately implemented.

Therefore, according to the embodiment, the model for estimating the psychological safety of the team may be accurately built on the basis of the post data communicated by the members in the team during work without a special user operation.

Note that the method for building the model described in the present embodiment of the invention may be implemented by causing a processor of a server or the like to execute a program prepared in advance. The present method is recorded on a computer-readable recording medium such as a hard disk, a flexible disk, a compact disc-read only memory (CD-ROM), a digital versatile disk (DVD), or a flash memory, and is read from the recording medium and executed by the computer.

Furthermore, the program may be distributed via a network such as the Internet.

All examples and conditional language provided herein are intended for the pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that the various changes, substitutions, and alterations could be made hereto without departing from the spirit and scope of the invention.

What is claimed is:

1. A method of building a model for estimating a level of psychological safety, the method comprising:
acquiring, by a computer, post data communicated between members in a team;
identifying fixed type post data that does not contribute to evaluation of the psychological safety among the acquired post data; and
creating the model based on content of the acquired post data from which the fixed type post data has been removed.

2. The method according to claim 1, further comprising:
identifying the fixed type post data based on similarity of content of a post in units of a contributor of post data or in units of a tool used for the post, and an occurrence frequency of post data.

3. The method according to claim 1, further comprising:
creating the model by
extracting a feature amount from the content for each of the members,
calculating a statistical value of the feature amounts for each team, and
performing a regression analysis by L1 regularization.

4. The method according to claim 3, further comprising:
calculating the statistical value including an interaction term of a combination of two different feature amounts.

5. The method according to claim 3, further comprising:
performing the regression analysis including input of a data aggregation value of a result of a questionnaire conducted in advance for the members, the questionnaire including question items regarding the estimation.

6. The method according to claim 3, further comprising:
extracting text information by converting non-text information included in the content into text.

7. The method according to claim 1, further comprising:
referring to the model created based on a predetermined period to be analyzed; and
outputting information regarding the psychological safety during the predetermined period.

8. A non-transitory computer-readable recording medium having stored therein a program that causes a computer to execute a process, the process comprising:
acquiring post data communicated between members in a team;
identifying fixed type post data that does not contribute to evaluation of psychological safety among the acquired post data; and
creating a model for estimating a level of the psychological safety based on content of the acquired post data from which the fixed type post data has been removed.

9. An information processing device, comprising:
a memory; and
a processor coupled to the memory and the processor configured to:
acquire post data communicated between members in a team;
identify fixed type post data that does not contribute to evaluation of psychological safety among the acquired post data; and
create a model for estimating a level of the psychological safety based on content of the acquired post data from which the fixed type post data has been removed.
